SEQUENCE LISTING (7 sequences)

SEQ ID NO: 1 - DNA, 2298 bp, Tripterygium wilfordii
  source 1..2298: mol_type = genomic DNA
atgtggaagataaagatagcggaacgtgggaactgtccctactccgagtacttgtacacgacgaacgacttcgttggtcgtcagatctgggagtttgacccgaacgcaggcacccccgaagaactggcggaggtcgaagaggctcgaaagaaattcaccgagaaccgccacgaagtcaagcctgcttccgaccttctttggatgcagcagtttctgaggcagaagaacttcaagcagaccattcctccgttgagaattggcgagaaagaacaagttacctatgaagacgtcacgaccgctttgaagatagcttcctcttttttttcagccctgcaggcctcggacggtcactggccagcagaaaatgctggagtcttgtttttccttcctccgttcattttctgcctatacatcacgggacatctcaattcgattatcacctcggaacaccgcaaggaaattctgcgcttcatatacaaccaccagaatgaggatgggggctggggaatacacatagagggacacagctcaatgtttaccacaacattcagctacgtctgcatgagaatcctgggagtaggaccagatgaaagtgcttgtgctagagcacgcaagtggattcttgaccacggtggtattacttacatggcttcctggggaaagacctggctttcgatacttggtatctttgattggtatggatgcaacccaatgcccccagagttctggattcttccctcttacctccctatacacccagcgaagatgtggtgttattgccggatggtctacatgcccatgtcttacctctatgggaaacggtttgttggccctataacacctctcattctgcaattgagagaggagctccacactcagccctaccatgagattcaatggcgtcagatgcgccatcgatgcggaaaggaagatctatactatccccactcgctcatccagaacttcctctgggactccctttatgtggcttccgaacctcttctgactcgctggcctttcagcaagataagagagaaggctcttgaaaaggcaatggaacacattcactacgaagacgaaaacagtcgttacatcaccattggctgtgtggaaaaggcattgtgcatgctttgttgctgggtggaagaccccaacggtgactatttcaagaagcatcttgcaaggatacccgattatctctgggttgccgaggatggtatgaaagtcgtgagtttcggctctcaattgtgggatgctacctttggctttcaagctctggttgctagcaacctaacaaacgaagtcgctcccaccctcgtcaaagcatacgacttcgtcaagaaatgccaggtccggaataacccctctggcgactttgagaaaatgttccgccacatttcaaaaggatcgtggaccttctccgaccaagatcatggctggcaactttccgactgcacggctgaggcactgaagtgttgcttgctagcagcaaccatgcccgaagaacttgtcggtgagaaattagatccccaatggatatttgagtctgtcaacatcatactatccctccaagagccgaaaaccggcggtcttgctggctgggaaccagtacgcgctggacaatggatggagatgctcaaccccatggagtttctcgagaacattgtgattgaacatacatatgtcgagtgcaccggatcctcaatcattgctttcattactctcaagaagctcttcccaggtcaccgaaccaaagacatcgacaatttcattgtcaacggtataagataccttgaagacgtccagtatcctgatggctcctggtacggaaactggggcatttgcttcatctatagtaccatgtttgcactgggaggcctagcagcagctggtaggacgtacgaaaattgtcccgcggtgcgaagaggcgttgactttttgcttattaatcagagtgatgatggtgggtggggtgaaagctatctttcttccccaagaaagatttatacaccccttgaaggaagaagatcaaatgtggtgcagactgcatgggctatgctggggcttctctatgctggccaggttgagcgcgaccctactcccctccaccgctgtgcgaaggtactgatcaattaccaaatggaagagggaggctacccccagcaggaaatcactggagttttcaaaatgaattgcatgctccactatccaatatatcgaaacgccttcccgatctgggcgctcggagagtaccggaaccgtgtcaagttgccttcgaaggaaattaagttgcaataa

SEQ ID NO: 2 - DNA, 20 bp, synthetic construct
  source 1..20: mol_type = other DNA
tcgttgatccggagagattc

SEQ ID NO: 3 - DNA, 20 bp, synthetic construct
  source 1..20: mol_type = other DNA
accagagcttgaaagccaaa

SEQ ID NO: 4 - DNA, 20 bp, synthetic construct
  source 1..20: mol_type = other DNA
agagcacgcaagtggattct

SEQ ID NO: 5 - DNA, 20 bp, synthetic construct
  source 1..20: mol_type = other DNA
atccggcaataacaccacat

SEQ ID NO: 6 - DNA, 21 bp, synthetic construct
  source 1..21: mol_type = other DNA
ctgggagctttactgcttgga

SEQ ID NO: 7 - DNA, 21 bp, synthetic construct
  source 1..21: mol_type = other DNA
atggctcgagatcgacgtaaa

(19) United States
(12) Patent Application Publication
(10) Pub. No.: US 2026/0152715 A1
(43) Pub. Date: Jun. 4, 2026

(54) Phaeodactylum tricornutum TwOSC4 mutant for High-Efficiency Friedelin Production

(71) Applicant: IUCF-HYU (INDUSTRY-UNIVERSITY COOPERATION FOUNDATION HANYANG UNIVERSITY), Seoul (KR)

(72) Inventors: EonSeon Jin, Seoul (KR); Khanh Nguyen, Seoul (KR)

(21) Appl. No.: 19/406,930

(22) Filed: Dec. 2, 2025

(30) Foreign Application Priority Data
Dec. 4, 2024 (KR) 10-2024-0178326
May 21, 2025 (KR) 10-2025-0066121

(51) Int. Cl. C12N 1/125; A61K 8/35; A61K 8/9706; A61K 31/122; A61K 36/02; A61Q 19/00; C12N 15/82; C12P 7/26; C12R 1/89

(52) U.S. Cl. CPC C12N 1/125; A61K 8/35; A61K 8/9706; A61K 31/122; A61K 36/02; A61Q 19/00; C12N 15/82; C12P 7/26; C12R 2001/89

(57) ABSTRACT

Provided is a Phaeodactylum tricornutum TwOSC4 mutant (Pt-HYU-OSC4) that produces friedelin, and makes it easier to culture and extract friedelin from the strain, and thus can increase the productivity of friedelin to mass-produce and industrially use friedelin. The mutant is produced through a transformation process, can be a transformant or a mutant, and is a strain that includes a TwOSC4 (oxidosqualene cyclase derived from Tripterygium wilfordii) gene, in which the gene is under an expression regulatory sequence that enables its expression, and that has been transformed into Phaeodactylum tricornutum.

### INCORPORATION OF SEQUENCE LISTING

This application contains a sequence listing submitted in Computer Readable Form (CRF). The CRF file contains the sequence listing entitled “10-PK003087808-SequenceListing.xml”, which was created on Dec. 2, 2025, and is 9,620 bytes in size. The information in the sequence listing is incorporated herein by reference in its entirety.

### CROSS-REFERENCE TO RELATED APPLICATION

This application claims priority to and the benefit of Korean Patent Application No. 2024-0178326, filed on Dec. 4, 2024, and Korean Patent Application No. 2025-0066121, filed on May 21, 2025, the disclosures of which are incorporated herein by reference in their entirety.

### BACKGROUND

### Field of the Invention

The present invention relates to a Phaeodactylum tricornutum TwOSC4 mutant (Pt-HYU-OSC4) for producing friedelin with high efficiency, and a method for producing friedelin using the same.

### Discussion of the Related Art

Friedelin is a critical biosynthetic precursor of celastrol isolated from Tripterygium wilfordii, and is a high-value-added substance with physiological activities such as anti-inflammatory, anti-hyperlipidaemic, and antidiabetic activities. Accordingly, friedelin is attracting attention from the pharmaceutical, functional food, and cosmetic industries.

Conventionally, friedelin has been extracted from the leaves, bark, and roots of certain plants (e.g., genus Celastrus and genus Quercus), but commercial production has been limited by low extraction yields and high costs.

Examples of cases where friedelin has been extracted from various plants are as shown in the table below.

| | Plant | | | |
|---|---|---|---|---|
| Plant | Material | Extraction conditions | Solvent | Friedelin conc. |
| Quercus | Cork | 120 mL solvent, 1 bar | Methanol | 12.1 wt % |
| cerris | | pressure, | Ethanol | 15.2 wt % |
| | | About 3 g biomass, 8 | Dichloromethane | 23.7 wt % |
| | | hours | Petroleum ether | 41.3 wt % |
| Quercus | Cork | No information | Dichloromethane | 26.03 wt % |
| cerris | | | | |
| Maytenus | Leaves | Extract 10 g of plant | Hexane | 0.49 wt % |
| aquifolium | | material with hexane and | | |
| | | chloroform for | | |
| | | consecutively 20 hours | | |
| Donbeya | Tree | Extract 1 kg of stem bark | Chloroform | NA |
| Torrida | bark | powder with chloroform | | |
| | | for 48 hours | | |
| Garcinia | Leaves | Extract 500 g of dried | n-post-hexane | 3.0 wt % |
| rubroechinata | | leave powder with post- | methanol | |
| | | hexane methanol for 24 | | |
| | | hours | | |
| Putranjiva | Leaves | Extract 50 g of dried | Chloroform | Leave extract: |
| roxburghii | and tree | powder with chloroform | | 0.003% w/w |
| | bark | for 6 hours | | Bark extract: |
| | | | | 0.04% w/w |

Plant-based friedelin extraction has problems of supply shortage and high cost, whereas chemical synthesis methods have disadvantages such as complex processes, extreme reaction conditions, and the production of toxic chemicals. Accordingly, it is urgent to develop an environmentally friendly and cost-effective method of producing friedelin.

To address these issues, biosynthetic approaches using microorganisms or microalgae have attracted increasing attention. Although microalgae are considered suitable metabolic engineering platforms due to their rapid growth, low cultivation costs, and ease of genetic manipulation, no microalgal strains capable of directly producing friedelin have yet been reported. While microalgae such as Chlamydomonas reinhardtii have been genetically engineered to produce terpenoids and other high-value-added compounds (Perozeni, F. et al., (2023), “Towards microalga-based superfoods: heterologous expression of zeolin in Chlamydomonas reinhardtii,” Frontiers in Plant Science, 14, 1184064), the biosynthesis of friedelin in microalgae remains entirely unexplored. Studies on the biosynthesis of friedelin using plant-derived oxidosqualene cyclase (OSC) genes have primarily been carried out in microbial expression systems such as Escherichia coli and Saccharomyces cerevisiae. Hansen et al. (Hansen, N. L. et al., Microbial Cell Factories, 19, 15 (2020)) demonstrated that TwOSC4 exhibits friedelin synthase activity in Nicotiana benthamiana; however, when TwOSC4 was introduced into yeast, production of friedelin was not observed. Meanwhile, no studies have reported friedelin biosynthesis in microalgae, nor has there been any case in which TwOSC4 has been expressed in microalgae to directly produce friedelin.

Accordingly, there is a need to develop a new transformant and production process capable of stably expressing the TwOSC4 gene and maximizing the yield of friedelin.

### SUMMARY OF THE INVENTION

The present inventors delivered a friedelin synthesis gene (oxidosqualene cyclase 4, OSC4) derived from a T. wilfordii plant into Phaeodactylum tricornutum to develop a new strain for friedelin production with easier cultivation and extraction.

An object of the present invention is to provide a Phaeodactylum tricornutum TwOSC4 mutant (Pt-HYU-OSC4) with accession number KCTC16288BP.

Further, another object of the present invention is to provide a method for producing the Phaeodactylum tricornutum TwOSC4 mutant, the method including: constructing an expression vector including the TwOSC4 gene and a Zeocin resistance marker under the control of an FcpB promoter; and

introducing the vector into Phaeodactylum tricornutum to generate a transformant.

In addition, still another object of the present invention is to provide a method for producing friedelin, the method including culturing the Phaeodactylum tricornutum TwOSC4 mutant.

Furthermore, yet another object of the present invention is to provide an oral composition including, as an active ingredient, one or more selected from the group consisting of a culture of the Phaeodactylum tricornutum TwOSC4 mutant, a dried product thereof, and an extract thereof, or friedelin isolated from the mutant.

Further, yet another object of the present invention is to provide a cosmetic composition including, as an active ingredient, one or more selected from the group consisting of a culture of the Phaeodactylum tricornutum TwOSC4 mutant, a dried product thereof, and an extract thereof, or friedelin isolated from the mutant.

In addition, yet another object of the present invention is to provide a food composition including, as an active ingredient, one or more selected from the group consisting of a culture of the Phaeodactylum tricornutum TwOSC4 mutant, a dried product thereof, and an extract thereof, or friedelin isolated from the mutant.

Furthermore, yet another object of the present invention is to provide a health functional food including, as an active ingredient, one or more selected from the group consisting of a culture of the Phaeodactylum tricornutum TwOSC4 mutant, a dried product thereof, and an extract thereof, or friedelin isolated from the mutant.

Hereinafter, the present invention will be described in more detail.

The present invention relates to a Phaeodactylum tricornutum TwOSC4 mutant with accession number KCTC16288BP.

Phaeodactylum tricornutum is a diatom, and a microalga found abundantly in marine environments and with high potential for bioengineering applications.

The Phaeodactylum tricornutum TwOSC4 mutant (accession number KCTC16288BP) strain according to the present invention is a mutant strain capable of producing friedelin.

The mutant is produced through a transformation process, may be a transformant or a mutant, and is a strain that includes a TwOSC4 (oxidosqualene cyclase derived from Tripterygium wilfordii) gene [SEQ ID NO: 1], in which the gene is under an expression regulatory sequence that enables its expression, and that has been transformed into Phaeodactylum tricornutum.

A deposit of the biological material, identified as Phaeodactylum tricornutum TwOSC4 mutant, Pt-HYU-OSC4, has been made with the Korean Collection for Type Cultures (KCTC), an International Depositary Authority under the Budapest Treaty, located at Korea Research Institute of Bioscience and Biotechnology (KRIBB), 181, Ipsin-gil, Jeongeup-si, Jeollabuk-do 56212, Republic of Korea. The deposit was made in April 2025 and assigned Accession Number KCTC 16288BP. The deposit will be maintained for at least thirty (30) years from the date of deposit and for at least five (5) years after the last request for the deposit, and will be made available to the public upon issuance of a U.S. patent, all in accordance with the Budapest Treaty and 37 C.F.R. §§ 1.801-1.809.

Further, the method for producing the Phaeodactylum tricornutum TwOSC4 mutant according to the present invention may include constructing an expression vector including the TwOSC4 gene and a Zeocin resistance marker under the control of an FcpB promoter, and introducing the vector into Phaeodactylum tricornutum to transform Phaeodactylum tricornutum.

As a specific example, the mutant may double every 20 to 36 hours (e.g., 24 hours) with an adequate amount of sunlight with an illuminance of 40 to 100 uE compared to other sources, and the Phaeodactylum tricornutum mutant of the present invention may reach a stationary growth state after only 7 days.

The Phaeodactylum tricornutum TwOSC4 mutant of the present invention may produce high amounts of friedelin, and thus the algae may be cultured to be effectively used as a raw material for foods, cosmetics, medicines, and the like.

In this regard, the present invention relates to a culture of the Phaeodactylum tricornutum TwOSC4 mutant.

In the present invention, “culture” refers to a medium in which a specific microorganism is cultured, that is, a post-culturing medium, and the culture is a culture including the Phaeodactylum tricornutum TwOSC4 mutant. Further, the culture is a culture including all of the concentrate of the culture where a post-culturing medium is subjected to processing such as concentration and drying, or the dried product of the culture. The culture can include a byproduct thereof, but the preparation thereof is not limited, and as an example, the culture may be a liquid or a solid.

The medium may contain nutritional components required by the microorganism to be cultured, that is, the microorganism that becomes the culture subject, in order to cultivate a specific microorganism, and may also be a medium in which additional components for special purposes are further added and mixed. The term “medium” also refers to a culturing medium or a culture solution, and encompasses natural media, synthetic media, and selective media A pH of the medium may be within a range in which the Phaeodactylum tricornutum TwOSC4 mutant can grow, and may be, for example, a pH of 6 or more, and preferably a pH of 6 to 9.

In addition, the present invention relates to a composition including, as an active ingredient, one or more selected from the group consisting of the Phaeodactylum tricornutum TwOSC4 mutant of the present invention, a culture of the algae, a dried product thereof, and an extract thereof, or friedelin produced from the mutant.

The Phaeodactylum tricornutum TwOSC4 mutant may be included as it is or in a dried form in the composition, and the culture of the algae may be included in a concentrated or dried form in the composition. Furthermore, the dried product is a dried form of the algae or the culture thereof, and may be in the form of a powder prepared by lyophilization, and the like.

Further, the extract is an extract obtained by extracting a product from the Phaeodactylum tricornutum TwOSC4 mutant of the present invention, a culture solution thereof, or a dried product thereof, and includes an extract using a solvent, and the like, and an extract obtained by disrupting the Phaeodactylum tricornutum TwOSC4 mutant of the present invention. Specifically, the extract may be an extract obtained by extracting and separating a pigment accumulated in the cells of the Phaeodactylum tricornutum TwOSC4 mutant of the present invention by a physical or chemical method.

The extraction process may be carried out by a typical method, and as an example, a target pigment may be extracted by adding an extraction solvent to the Phaeodactylum tricornutum TwOSC4 mutant of the present invention, homogenizing the resulting mixture, and then disrupting the microbial cells. After the extraction, a disrupted product of the algae may be removed through centrifugation, and the extraction solvent may be removed by a method such as distillation under reduced pressure. In addition, the extraction procedure may further include a typical purification process.

Since the Phaeodactylum tricornutum TwOSC4 mutant of the present invention has an excellent friedelin production ability, a composition including the mutant and by-products thereof such as a culture, a dried product, and an extract has antioxidant, anti-inflammatory, anti-cancer, antibacterial/anti-fungal, hepatoprotective, neuroprotective, and antidiabetic preventive or therapeutic effects. The composition of the present invention may be used as a raw material included in foods, health functional foods, medicines, feeds, and the like for maintaining physical health.

The composition may be used to improve human and animal health, and may be a cosmetic composition. The cosmetic composition may have antioxidant and anti-inflammatory effects.

The cosmetic composition may be prepared as any formulation typically prepared in the art, and may be formulated as, for example, a solution, a suspension, an emulsion, a paste, a gel, a cream, a lotion, a powder, a soap, a surfactant-containing cleanser, an oil, a powdered foundation, an emulsion foundation, a wax foundation, a spray, or the like, but is not necessarily limited thereto. More specifically, the cosmetic composition may be formulated into a cosmetic of formulations such as softening lotions, gels, astringent lotions, nourishing lotions, massage creams, essences, eye creams, eye essences, cleansing creams, cleansing foams, cleansing waters, packs, powders, body lotions, body oils, mists and body essences, water-soluble liquids, milk lotions, creams, skin toners, oil-in-water emulsions, water-in-oil emulsions, oil-in-water makeup bases, water-in-oil makeup bases and foundations, and may be applied to the skin or may be absorbed into the skin using microneedles or the like.

In the cosmetic composition of the present invention, pharmaceutically or cosmetically acceptable carriers vary depending on the formulation thereof, but examples thereof include hydrocarbons such as petrolatum, liquid paraffin, and gelled hydrocarbons (also known as Plastibase); animal and vegetable oils such as medium-chain fatty acid triglycerides, lard, hard fat, and cocoa butter; higher fatty acid alcohols such as cetanol, stearyl alcohol, stearic acid, and isopropyl palmitate, and fatty acids and esters thereof; water-soluble bases such as Macrogol (polyethylene glycol), 1,3-butylene glycol, glycerol, gelatin, sucrose, and sugar alcohols; emulsifiers such as glycerine fatty acid ester, stearic acid polyoxyl, and polyoxyethylene cured castor oil; adhesives such as acrylic acid ester and sodium alginate; propellants such as liquefied petroleum gas and carbon dioxide; preservatives such as paraoxybenzoic acid esters, and the like, and the external preparation of the present invention may be prepared by typical methods using these. Furthermore, in addition to these, a stabilizer, a fragrance, a colorant, a pH adjuster, a diluent, a surfactant, a preservative, an antioxidant, and the like may also be blended as needed. The external preparation of the present invention may be applied to a local wound area by a typical method.

Further, the composition may be a composition for oral administration, in that the composition may be supplied via an oral route included in a food, a medicine, a feed, or the like.

The composition for oral administration may be included in a formulated oral preparation by using a method publicly known in the art, such as a powder, a granule, a tablet, a pill, a sugar-coated tablet, a liquid, a gel, a syrup, a slurry, and a suspension. For example, for the oral preparation, a tablet or a purified material of sugar may be obtained by blending an active ingredient with a solid excipient, pulverizing the same, adding a suitable auxiliary agent thereto, and then processing the same into a granular mixture. Examples of a suitable excipient include sugars including lactose, dextrose, sucrose, sorbitol, mannitol, xylitol, erythritol, maltitol, and the like, starches including corn starch, wheat starch, rice starch, potato starch, and the like, celluloses including cellulose, methyl cellulose, sodium carboxymethyl cellulose, hydroxypropylmethyl-cellulose, and the like, and fillers such as gelatin and polyvinylpyrrolidone. In addition, a crosslinked polyvinylpyrrolidone, agar, alginic acid, sodium alginate, or the like may be added as a disintegrating agent in some cases.

Furthermore, the composition can be added in order to achieve a purpose use which is special for a food, and thus may be a food composition or a composition for a food in this regard. When the composition is used in food, it is produced by the Phaeodactylum tricornutum TwOSC4 mutant, and friedelin in the mutant may maintain or enhance the health of the body. Specifically, the friedelin is a naturally occurring compound belonging to the triterpenoid group, and is effective in preventing or improving antioxidant, anti-inflammatory, anti-cancer, antibacterial/anti-fungal, hepatoprotective, neuroprotective, and antidiabetic properties and the like. Because of these effects, the food composition may be used to prevent or ameliorate the symptoms, or for the effects.

In the present invention, “for an additive” includes any food composition as long as the food composition is a constitution in which ingredients other than the main ingredient are added to a food, and a specific example thereof may be an effectively active material having functionality in a food or feed or a food additive defined by the Ministry of Food and Drug Safety of the Republic of Korea to be added for coloring, preservation, and the like in a processed food.

The food composition of the present invention may include ingredients typically added during the production of food, and includes, for example, a protein, carbohydrate, fat, nutrient, seasoning, and flavoring agent. Examples of the above-described carbohydrate include typical sugars such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, and erythritol. As the flavoring agent, it is possible to use a natural flavoring agent [thaumatin, stevia extract (for example, rebaudioside A, glycyrrhizin and the like)] and a synthetic flavoring agent (saccharin, aspartame, and the like). For example, when the food composition of the present invention is prepared as a drink or a beverage, the composition may additionally include citric acid, liquid fructose, sugar, sucrose, acetic acid, malic acid, a fruit juice, various vegetable extracts, and the like.

The food composition may additionally include food additives, and unless otherwise stipulated, whether or not an item is suitable as a “food additive” is determined according to the specifications and criteria for the item in question, in accordance with the General Rules and General Test Methods for Food Additives Code, and the like approved by the Ministry of Food and Drug Safety.

Examples of products listed in the “Food Additive Code” include chemically synthesized products such as ketones, glycine, potassium citrate, nicotinic acid, and cinnamic acid, natural additives such as a persimmon color, licorice extract, crystalline cellulose and guar gum, and mixed preparations such as L-sodium glutamate preparations, alkaline preparations added to noodles, preservative preparations, and tar color preparations.

Examples of a food including the active ingredient of the present invention include confectionary such as bread, rice cakes, dried fruit, candy, chocolate, chewing gum, and jam, ice cream products such as ice cream, frozen desserts, and ice cream powder, dairy products such as milk, low-fat milk, lactose-digested milk, processed milk, goat milk, fermented milk, buttermilk, condensed milk, milk cream, butter oil, natural cheese, processed cheese, powdered milk, and whey, meat products such as processed meat products, egg products, and hamburger, fish and meat products including processed fish and meat products such as fish cakes, ham, sausage, and bacon, noodles such as instant noodles, dried noodles, raw noodles, instant fried noodles, gelatinized dry noodles, modified cooked noodles, frozen noodles, and pasta, beverages such as fruit drinks, vegetable drinks, carbonated drinks, soy milk, lactic acid bacteria beverages such as yogurt, and mixed drinks, seasonings such as soy sauce, soybean paste, red pepper paste, black bean paste, cheonggukjang, mixed soy paste, vinegar, sauces, tomato ketchup, curry, and dressing, margarine, shortening, and pizza, but are not limited thereto.

In addition to the aforementioned ingredients, the composition of the present invention may include various nutrients, vitamins, electrolytes, flavors, colorants, pectic acids and salts thereof, alginic acid and salts thereof, protective colloid thickeners, pH adjusters, stabilizers, preservatives, glycerin, alcohols, carbonation agents used in carbonated beverages, and the like. In addition, the composition of the present invention may include flesh for preparing natural fruit juice, fruit juice drinks, and vegetable drinks. These ingredients may be used either alone or in combination.

The beverage composition including the active ingredient of the present invention is not particularly limited in terms of other ingredients, and may contain various flavoring agents, natural carbohydrates, and the like as additional ingredients as in a typical beverage. Examples of the above-described natural carbohydrates include typical sugars such as monosaccharides, (for example, glucose, fructose and the like); disaccharides, (for example, maltose, sucrose and the like); and polysaccharides, (for example, dextrin, cyclodextrin and the like), and sugar alcohols such as xylitol, sorbitol, and erythritol. As the flavoring agent other than those mentioned above, a natural flavoring agent (thaumatin, a stevia extract (for example, rebaudioside A, glycyrrhizin and the like)), and a synthetic flavoring agent (saccharin, aspartame and the like) may be advantageously used.

The food may be a health functional food. More specifically, the food may be a health functional food for anti-inflammatory and antioxidant purposes.

As used herein, the term “health functional food” refers to food manufactured and processed using raw materials or ingredients that have functionality useful for the human body, in accordance with the Health Functional Foods Act (Article 3, Paragraph 1), and the “functionality” refers to regulating nutrients to the structure and function of the human body, or obtaining effects useful for health purposes such as physiological actions (Article 3, Paragraph 2).

Furthermore, the composition may be mixed with a carrier and a fragrance typically used in the food or pharmaceutical field and may be prepared and administered in the form of a tablet, a troche, a capsule, an elixir, a syrup, a powder, a suspension, a granule, or the like. As the carrier, it is possible to use a binder, a lubricant, a disintegrating agent, an excipient, a solubilizing agent, a dispersing agent, a stabilizing agent, a suspending agent, and the like. As an administration method, an oral, parenteral, or application method may be used, but the composition is preferably orally administered. In addition, the administered dose may be appropriately selected depending on the absorption degree, the inactivation rate and the excretion rate of an active ingredient in the body, and age, gender, condition, and the like of a person receiving the administration. A pH of the composition can be easily changed depending on the manufacturing conditions and the like of the medicine, food, and the like in which the composition is used.

The pharmaceutical composition may be preferably formulated as a pharmaceutical composition by including one or more pharmaceutically acceptable carriers in addition to the active ingredient described above for administration.

For example, for formulation into a tablet or capsule, the active ingredient may be combined with an oral, non-toxic, pharmaceutically acceptable inert carrier such as ethanol, glycerol, and water. When desired or necessary, a suitable binder, lubricant, disintegrating agent and colorant may also be included in the mixture. Suitable binders include, but are not limited to, natural sugars such as starch, gelatin, glucose or β-lactose, natural and synthetic gums such as corn sweetener, acacia, tragacanth or sodium oleate, sodium stearate, magnesium stearate, sodium benzoate, sodium acetate, sodium chloride, and the like. Disintegrating agents include, but are not limited to, starch, methyl cellulose, agar, bentonite, xanthan gum, and the like. In a composition formulated as a liquid solution, a pharmaceutically acceptable carrier may be suitable for sterilization, and in a living body, it is possible to use saline, sterilized water, Ringer's solution, buffered saline, albumin injection, a dextrose solution, a malto dextrin solution, glycerol, ethanol, and a mixture of one or more thereof, and other typical additives, such as an antioxidant, a buffer, and a bacteriostatic agent may be added, if necessary. Further, the composition may be formulated into a dosage form for injecting, a pill, a capsule, a granule or a tablet, such as an aqueous solution, a suspension, and an emulsion by additionally adding a diluent, a dispersant, a surfactant, a binder, and a lubricant.

The composition may include friedelin or any one selected from the group consisting of a Phaeodactylum tricornutum TwOSC4 mutant, a culture of the mutant, a dried product thereof, and an extract thereof in an amount of 0.001 to 99.99 wt %, preferably 0.1 to 99 wt %, based on the total weight of the composition, and the content of an active ingredient may be appropriately adjusted depending on the method for using the composition and the purpose of using the composition.

In addition, another object of the present invention is to provide a method for producing friedelin using the Phaeodactylum tricornutum TwOSC4 mutant of the present invention.

Furthermore, still another object of the present invention is to provide a method for producing a food raw material or friedelin, the method including culturing the Phaeodactylum tricornutum TwOSC4 mutant of the present invention.

When the Phaeodactylum tricornutum TwOSC4 mutant of the present invention is used, a content of friedelin in the algae to be cultured may be increased, so that the supply of a raw material for industrial use and the like may be efficiently carried out.

The production method may include culturing the Phaeodactylum tricornutum TwOSC4 mutant.

Further, the production method may further include: separating the Phaeodactylum tricornutum TwOSC4 mutant of the present invention from the culture after the culturing of the Phaeodactylum tricornutum TwOSC4 mutant of the present invention. The separated algae may be further subjected to a processing step including drying.

In addition, the production method may further include: extracting friedelin from the Phaeodactylum tricornutum TwOSC4 mutant of the present invention, a culture of the mutant, a concentrate of the culture, or a dried product of the culture.

The culturing may be carried out at an illumination of 40 to 100 uE at 18 to 25° C. for 4 to 8 days. The culturing may be carried out in a medium under a pH condition of 6.0 to 8.0. The culturing medium is easily prepared similarly to seawater conditions, and preferably F/2 medium (ASW-artificial sea water) is used.

The culturing is carried out in F/2 medium under a 12:12 photoperiod, and the strain is harvested after 4 to 8 days of culture.

The culturing may be carried out in a medium under a pH condition of 6.0 to 8.0. Furthermore, the culturing may be carried out under dim light conditions, specifically, under light intensity conditions within a range of 40 to 100 μmol photons/m²s (μE).

Since the Phaeodactylum tricornutum TwOSC4 mutant of the present invention has an excellent friedelin production ability even at low light intensities, the mutant may be produced highly efficiently without inputting high-intensity light energy, and thus may be industrially effectively used.

The extraction described above is preferably an ultrasonic extraction method for extracting friedelin from microorganisms.

The ultrasonic extraction method involves extracting friedelin with an organic solvent using ultrasound. The organic solvent may be one or more selected from the group consisting of methanol, ethanol, chloroform, hexane, dichloromethane, and petroleum ether, but methanol is most preferred.

The preparation method may further include, in addition to the culturing step, a concentrating step of increasing the content of the algae after the culturing and a drying step of drying the algae subjected to the concentrating step by further reducing moisture in the algae. However, the concentrating step or the drying step is not necessarily needed, and may be generally carried out by using a concentrating and drying method, and a machine typically used in the field to which the present invention belongs.

The preparation method may further include a purification step after the extracting step, and the purification step may be carried out by a typical purification method in the field to which the present invention belongs.

Friedelin prepared through the concentrating or drying step may be used as a raw material for a food, a health functional food, a cosmetic, a medicine, or the like.

In one embodiment of the present invention, friedelin produced from the novel strain Phaeodactylum tricornutum TwOSC4 mutant may be obtained in an amount of 50 ng/mL to 60 ng/ml (52 ng/ml to 58.4 ng/ml).

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 illustrates the optimized gene sequence of TwOSC4;

FIG. 2 illustrates the TwOSC4 expression vector cleavage map;

FIGS. 3A and 3B illustrate the colony PCR results for the Phaeodactylum tricornutum TwOSC4 mutant (Pt-HYU-OSC4) [A) TwOSC4 expression cassette in a pFcpB-TwOSC4-Ble vector and B) genomic PCR results for wild-type (WT) and Pt-OSC4 strains (Pt-HYU-OSC4-20 and 48), the upper panel illustrates the results of fcpB::TwOSC4 PCR for vector sequence detection using fcpB-Fw and TwOSC4-Rv primers, and the lower panel illustrates the control of DNA samples confirmed by ITS region PCR using internal transcribed spacer (ITS) primers];

FIG. 4 illustrates the results of analyzing TwOSC4 gene expression in Pt-HYU-OSC4 through qRT-PCR [Relative mRNA expression levels of TwOSC4 in the WT and mutants Pt-OSC4-20 and Pt-OSC4-48; tubulin A (TubA): control; error bars indicate an average value obtained from three independent experiments];

FIG. 5 illustrates the SDS-PAGE gel results for total protein extraction and the immunoblotting (western blot) results for TwOSC4 protein expression analysis in the WT [left] and mutants Pt-OSC4-20 and Pt-OSC4-48 [right];

FIG. 6 illustrates the growth curves of the Phaeodactylum tricornutum WT and five candidate TwOSC4 mutants [cells were cultured in F/2 medium];

FIGS. 7A and 7B illustrate the production analysis of friedelin extracted from two candidate Phaeodactylum tricornutum TwOSC4 mutants as measured by LC-MS [1:5 methanol: culture solution volume ratio];

FIGS. 8A and 8B illustrate the friedelin production analysis results according to RO 48-8071 (fumarate-PtOSC suppressor) in the Phaeodactylum tricornutum TwOSC4 mutant Pt-OSC4-20, as measured by LC-MS [1:1 methanol: culture solution volume ratio] [in the LC-MS results, the first peak corresponds to friedelin, and the second peak corresponds to fumarate]; and

FIG. 9 illustrates the mevalonate (MVA) pathway and sterol biosynthesis pathway in Phaeodactylum tricornutum, and the friedelin expression pathway in the transformed strain [relevant key enzymes are in bold, and chemical enzyme inhibitors are underlined; acetyl-coenzyme A (ACCOA); aceto-acetyl-coenzyme A (AACCOA); 3-hydroxy-3-methylglutaryl-coenzyme A (HMGCOA); MVA; mevalonate phosphate (MVAP); mevalonate diphosphate (MVAPP); isopentenyl diphosphate (IPP); dimethylallyl diphosphate (DMAPP); geranyl diphosphate (GPP); farnesyl diphosphate (FPP); presqualene diphosphate (PSDP); squalene epoxidase (SQE); Phaeodactylum tricornutum's oxidosqualene cyclase (PtOSC); Tripterygium wilfordii's Oxidosqualene cyclase 4 (TwOSC4); Fumarate (Ro 48-8071)].

### DETAILED DESCRIPTION OF EXEMPLARY EMBODIMENTS

Hereinafter, the present invention will be described in detail through examples. However, the following examples are only for exemplifying the present invention, and the scope of the present invention is not limited to the following examples.

### Examples

### Example 1: Culturing of Phaeodactylum tricornutum Mutant

Diatom cells were cultured in F/2 medium including silicate (+Si) (ASW) in 40 mM Tris-HCl buffer (pH 7.2) in a shaking incubator (130 rpm). The culturing of the Phaeodactylum tricornutum mutant was maintained at 20 to 22° C. and set at a 12:12 light:dark cycle. Light was provided by a fluorescent lamp (about 50 μmol/m²s). The medium was supplemented with 10 mM sodium bicarbonate in order to provide inorganic carbon advantageous in cell culture.

TABLE 1
F/2 medium composition

| F/2 medium (ASW) component | Concentration | |
|---|---|---|
| Sea salt | 32 | g/L |
| NaNo₃ | 880 | μM |
| NaH₂PO₄—H₂O | 36 | μM |
| Na2SiO3—9H2O | 106 | μM |

Trace metal solution (1 mL/L)

| | | |
|---|---|---|
| ZnSO₄—7H₂0 | 0.08 | μM |
| MnSO₄—H₂O | 0.9 | μM |
| Na₂MoO₄—2H₂O | 0.03 | μM |
| CoSO₄—7H₂O | 0.05 | μM |
| CuCl₂—2H₂O | 0.04 | μM |
| Fe(NH₄)₂(SO₄)₂—6H₂O | 11.7 | μM |
| Na₂EDTA—2H₂0 | 11.7 | μM |

Vitamin solution (1 mL/L)

| | | |
|---|---|---|
| Vitamin B₁₂ | 0.1 | mM |
| Biotin Vitamin | 0.1 | mM |
| Thiamine Vitamin | 1 | mM |

### Example 2: Construction of TwOSC4 Expression Vector

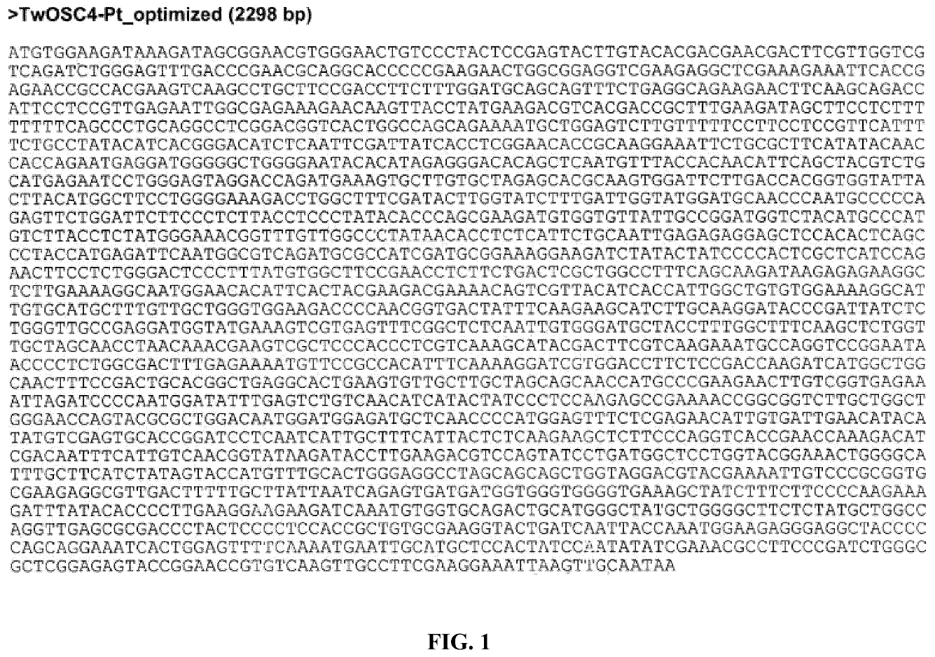

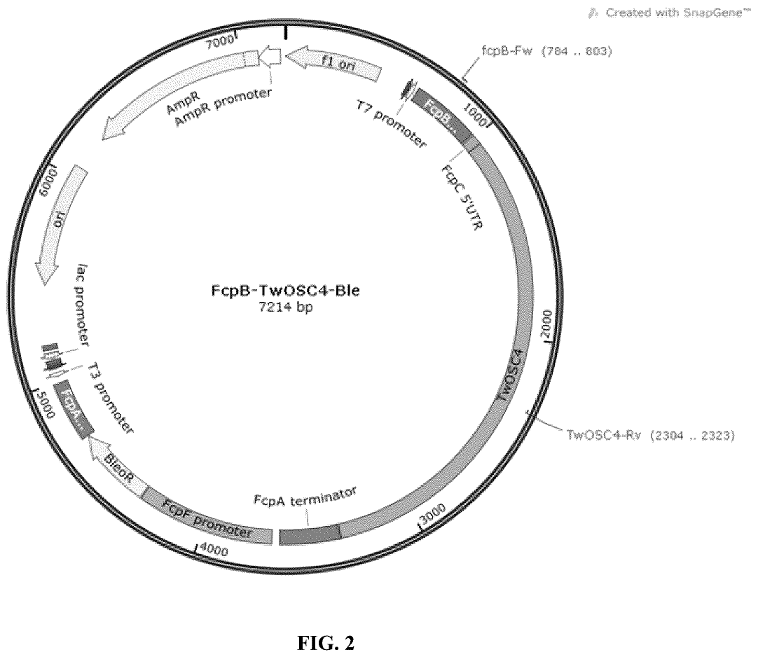

An expression vector (FIG. 2) for the TwOSC4 gene (FIG. 1) was constructed using an FcpB promoter.

The codons of the TwOSC4 gene were derived from plant genomes and optimized for successful expression in diatoms. The TwOSC4 gene expression vector was constructed from a previously designed plasmid pPhaT-EF2-Luc [Seo, S., Jeon, H., Hwang, S., Jin, E., & Chang, K. S. aureus Development of a new constitutive expression system for the transformation of the diatom Phaeodactylum tricornutum. Algal Research, 11, 50-54 (2015)].

The PPhaT-EF2-Luc plasmid was digested with XbaI and SpeI to remove a fragment encoding luciferase. Thereafter, the TwOSC4 gene was cloned into pphaT-EF2-Luc, in which the luciferase gene had been deleted.

This vector includes an fcpB promoter of Phaeodactylum tricornutum, which is a strong promoter derived from a fucoxanthin chlorophyll a/c-binding protein B gene encoding a component of a light-harvesting complex. A bleomycin resistance gene was used as a resistance marker to Zeocin antibiotic.

### Example 3: Transformation and Screening of Phaeodactylum tricornutum for PtHYU-OSC4 Candidates

The plasmid construct (FIG. 2) was introduced by biolistic bombardment into Phaeodactylum tricornutum using coated tungsten particles. M17 (1.1 μm diameter) tungsten particles were coated with the plasmid, and 5×10⁷ Phaeodactylum tricornutum cells in the exponential growth phase were cultured in the center of F/2+Si 1.2% agar medium. Particle bombardment was performed using Biolistic Particle Delivery System PDS-1000/He (Bio-Rad Laboratories, CA, USA), and a 1550 psi rupture disk was mounted according to the manufacturer's recommendations (BIO-RAD 1,550 psi Rupture Disks #1652331). The cells were aliquoted onto F/2 medium agar plates including 100 μg/mL Zeocin, and resistant colonies were selected for 2 to 3 weeks. The colonies were transferred to new plates, and well-grown transformants were selected by colony PCR.

Primers for detection of the exogenous FcpB::TwOSC4 gene were 5′-TCGTTGATCCGGAGAGATTC-3′ (Fw, SEQ ID NO: 2) and 5′-ACCAGAGCTTGAAAGCCAAA-3′ (Rv, SEQ ID NO: 3) (product size=1540 bp).

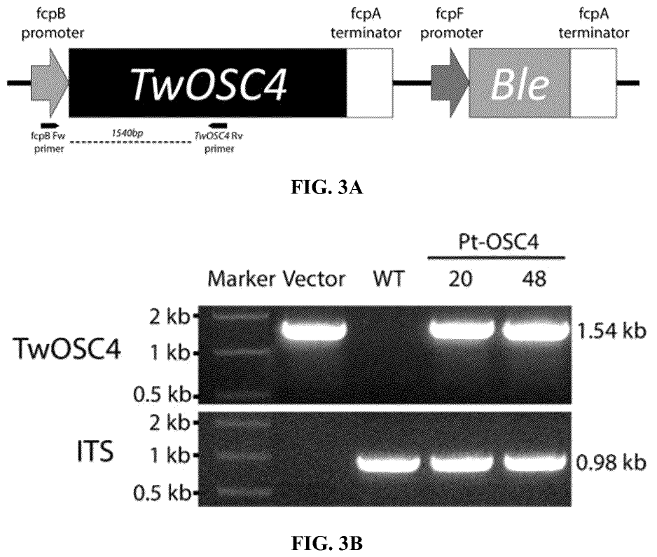

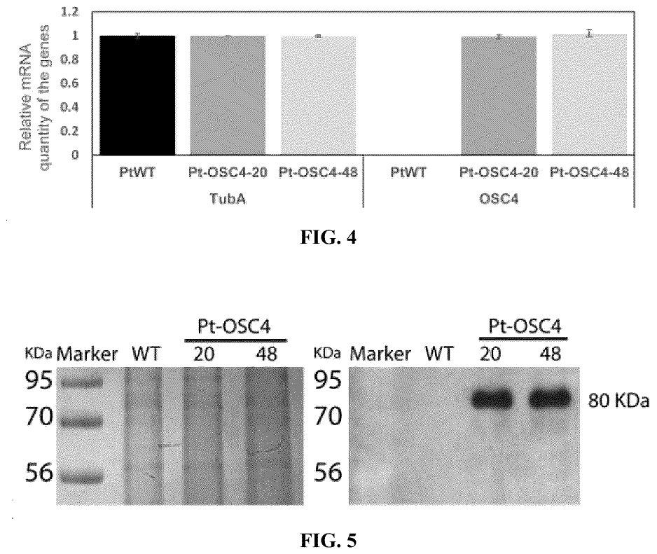

A plasmid template was used as a positive control, and DNA from the wild-type strain (PtWT) was used as a negative control (FIGS. 3A-3B).

Total RNA was purified 5 hours after exposure to light and used for quantitative real-time PCR (qRT-PCR) analysis to measure the relative expression levels of the TwOSC4 gene in the wild type and two transformed strains (Pt-OSC4-20 and 48). All values are presented as the mean and standard deviation of two technical replicates for independently prepared biological samples (n=3).

Primers used for qRT-PCR for TWOSC4 gene were 5′-AGAGCACGCAAGTGGATTCT-3′ (Fw, SEQ ID NO: 4) and 5′-ATCCGGCAATAACACCACAT-3′ (Rv, SEQ ID NO: 5).

Alpha tubulin (TubA) was amplified as a positive control, and primers used were 5′-CTGGGAGCTTTACTGCTTGGA-3′ (Fw, SEQ ID NO: 6) and 5′-ATGGCTCGAGATCGACGTAAA-3′ (Rv, SEQ ID NO: 7).

### Example 4: Immunoblotting (Western-Blot Analysis)

A culture solution was harvested by centrifugation at 2,000×g for 15 min in a centrifuge. A precipitate was suspended in extraction buffer (10 mM Tris-HCl, 1 mM EDTA, 0.2% SDS) and 1× protease inhibitor mixture (Thermo Scientific, IL, USA). Cells were disrupted by sonication (irradiated with ultrasound for 30 seconds each for four cycles, with a 30-second rest period between cycles, and the ultrasound intensity was 285 W). The total extracted protein was measured using a Pierce BCA protein assay kit (Thermo Scientific) and loaded onto a 10% SDS-PAGE gel (20 μg per lane). The gel was stained with Coomassie blue to confirm equivalent loading. Alternatively, the separated protein (friedelin synthase TwOSC4) was electrotransferred to a PVDF membrane and subjected to immunoblotting. A primary antibody against Friedelin synthase TwOSC4 (Thermo Scientific, USA) was used at a dilution of 1:1,000. As illustrated in FIG. 5, TwOSC4 protein was not detected in the WT strain.

### Example 5: Cell growth and friedelin production

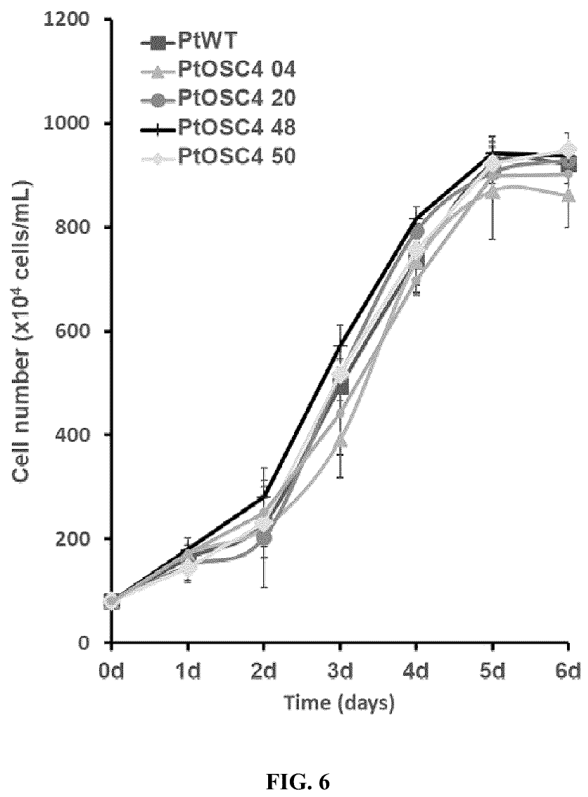

The WT and candidate mutants were inoculated at the same cell density, about 1.0×10⁶ cells/mL, and cell growth was measured by cell density. The number of cells was estimated by counting cells using a Neubauer chamber. After initial inoculation, cells showed an exponential growth until day 5 of culture, and then cell growth entered a stationary state. According to the results, the growth curves of WT and mutants PtHYU-OSC4-04,-20,-48, and -50 were very similar. 6 days after inoculation, the cell density of the WT reached 9.23±0.58×10⁶ cells/mL, the cell numbers of the mutants PtHYU-OSC4-04,-20,-48, and -50 were 8.62±0.61×10⁶ cells/mL, 9.3±0.15×10⁶ cells/mL, 9.36±0.18×10⁶ cells/mL, and 9.5±0.18×10⁶ cells/mL, respectively, and no statistically significant differences in growth were observed (FIG. 6).

The production amount of friedelin by Pt-HYU-OSC4-20 was evaluated as follows. For detection, 10 mL of cultured cells were harvested by centrifugation at 1300 rpm for 15 minutes. After removing all the media, a pellet was suspended in 2 mL of 100% methanol and stored in a sonication bath for 30 seconds. The pellet was centrifuged at 1300 rpm for 5 minutes to collect a supernatant, and the supernatant was filtered through a 0.2 μm Nylon Whatman filter, and then measured by LC-MS.

The production amount of friedelin was calculated according to the magnitude of the LC-MS measurement signal.

A liquid chromatography quadrupole time-of-flight mass spectrometry (LC-QTOF/MS) system was composed of an Agilent 1290 Infinity HPLC system equipped with an electrospray ionization source and an AB sciex TripleTOF 5600 Plus mass analyzer. As an analytical column, Poroshell120 (2.1 mm×50 mm, 2.7 μm, Agilent Technologies, California, USA) was used. The column temperature was maintained at 35° C. A reverse HPLC system was performed using an isokinetic system with a mobile phase of acetonitrile:methanol (30:70, v/v) and a flow rate of 0.3 mL/min. The injection amount was 5 μL. Mass was detected in positive ion mode using parameters such as curtain gas 25 psi, drying temperature 500° C., ion spray voltage 4,500 V, and collision gas helium.

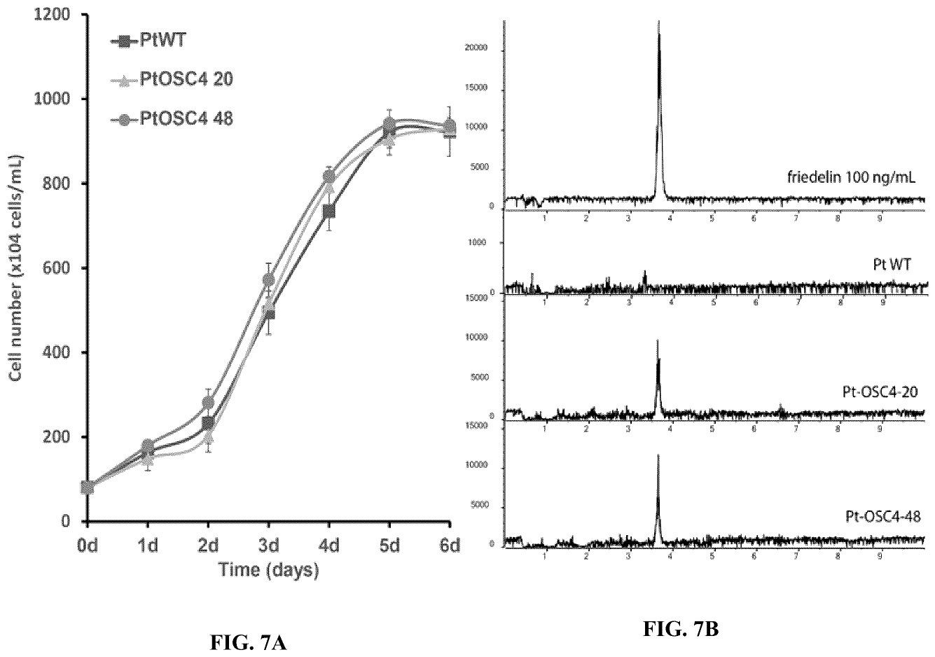

The production of friedelin in PtHYU-OSC4-20 and PtHYU-OSC4-48 was confirmed through liquid chromatography-mass spectrometry (LC-MS). For friedelin detection, 10 mL of the culture solution was harvested on day 6 by centrifugation at 1,300 rpm for 15 minutes. After removing all the media, a pellet was suspended in 2 mL of 100% methanol and then treated in a sonication bath for 30 seconds. Thereafter, the pellet was centrifuged again at 1,300 rpm for 5 minutes to collect a supernatant, and the supernatant was filtered through a 0.2 μm Nylon Whatman filter before LC-MS analysis. A friedelin standard was purchased from Sigma Aldrich (Seoul, Korea). As a result, PtHYU-OSC4-20 and PtHYU-OSC4-48 could produce up to 58.4 ng/ml and 57.2 ng/ml of friedelin, respectively (FIGS. 7A-7B).

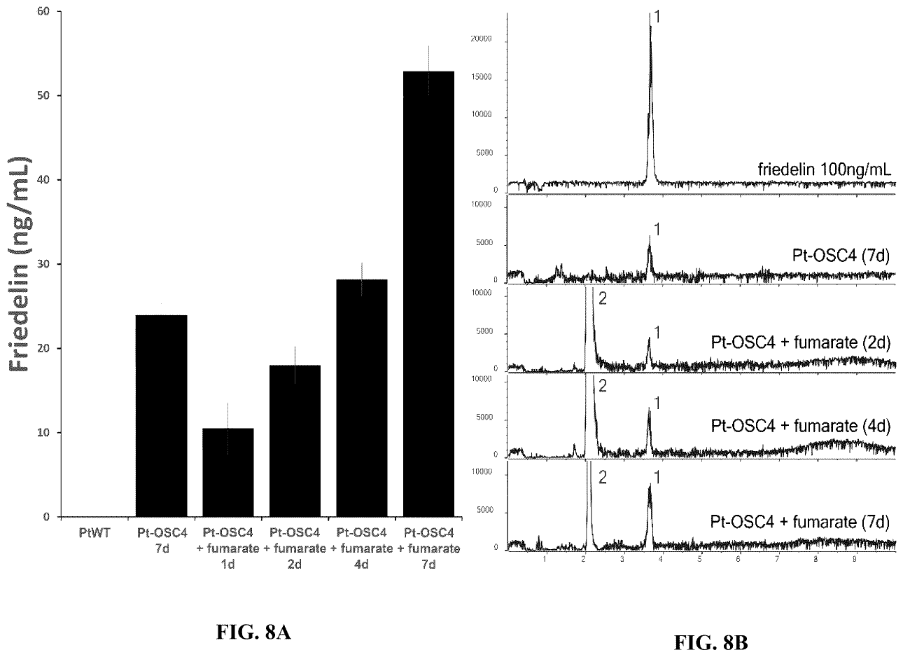

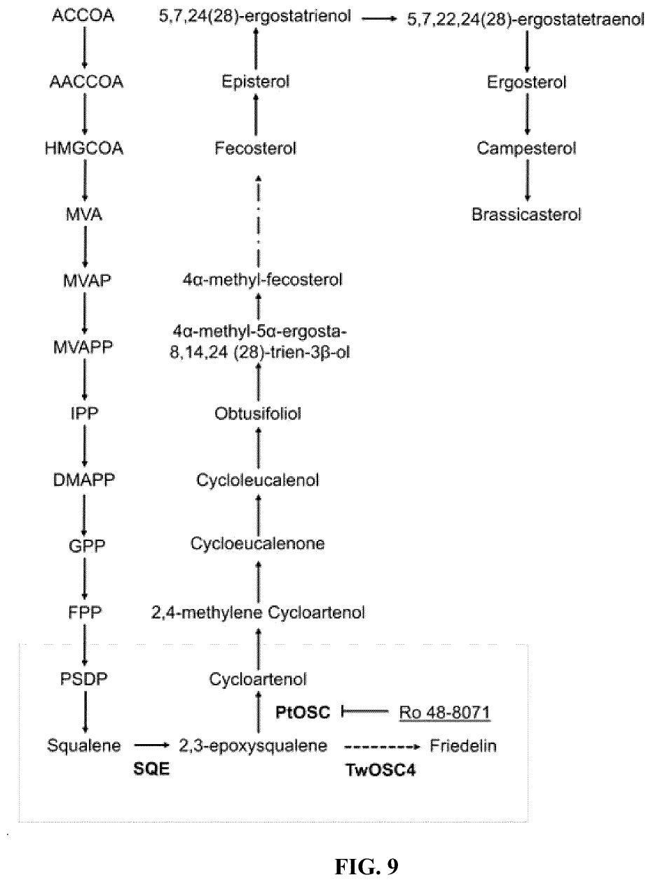

The results of LC-MS analysis of Pt-OSC4-20 treated with fumarate (RO 48-8071, a PtOSC suppressor) are shown in FIGS. 8A-8B. In this case, friedelin was extracted by setting a volume ratio of methanol and culture medium to 1:1, unlike FIGS. 7A-7B which show the results of extracting methanol and culture solution at a volume ratio of 1:5.

Suppression of PtOSC activity doubled the accumulation amount of friedelin, reaching a maximum of 55 ng/mL after 7 days compared to 26 ng/mL under general F/2 medium conditions. This means that genome editing techniques, such as PtOSC gene deletion and SQE gene overexpression, could be applied in order to improve friedelin production in the future.

The selected strain, PtHYU-OSC4-20, was named Phaeodactylum tricornutum TwOSC4 mutant, Pt-HYU-OSC4, deposited with the Korean Collection for Type Cultures (KCTC) at Korea Research Institute of Bioscience and Biotechnology (KRIBB) on Apr. 1, 2025, and assigned the accession number KCTC 16288BP.

The microalgae of the present invention, particularly the diatom Phaeodactylum tricornutum TwOSC4 mutant, are expected to not only simplify the process compared to existing sources, but also greatly improve friedelin production, thereby enabling mass cultivation and industrial use.

While the aforementioned description merely exemplarily describes the technical spirit of the present invention, a person with ordinary skill in the art to which the present invention pertains can make various modifications and alterations within the scope not departing from the essential characteristics of the present invention. Accordingly, the embodiments disclosed in the present invention are intended not to limit but to describe the technical spirit of the present invention, and the scope of the technical spirit of the present invention is not limited by these embodiments. The protection scope of the present invention must be interpreted by the following claims and it should be interpreted that all technical ideas within a scope equivalent thereto are included in the scope of rights of the present invention.

1. A Phaeodactylum tricornutum TwOSC4 mutant (Pt-HYU-OSC4) with accession number KCTC16288BP.

2. The Phaeodactylum tricornutum TwOSC4 mutant of claim 1, wherein the mutant produces friedelin.

3. The Phaeodactylum tricornutum TwOSC4 mutant of claim 1, wherein the mutant has a TwOSC4 gene represented by SEQ ID NO: 1.

4. A method for producing the Phaeodactylum tricornutum TwOSC4 mutant of claim 1, the method comprising: constructing an expression vector including the TwOSC4 gene and a Zeocin resistance marker under a control of an FcpB promoter; and
introducing the vector into Phaeodactylum tricornutum to transform Phaeodactylum tricornutum.

5. A method for producing friedelin, the method comprising culturing the Phaeodactylum tricornutum TwOSC4 mutant of claim 1.

6. The method of claim 5, wherein the culturing is carried out at an illuminance of 40 to 100 μE and 18 to 25° C. for 4 to 8 days.

7. The method of claim 5, further comprising, after the culturing, extracting friedelin with an organic solvent using ultrasound.

8. The method of claim 7, wherein the organic solvent is one or more selected from the group consisting of methanol, ethanol, chloroform, hexane, dichloromethane, and petroleum ether.

9. An oral composition comprising, as an active ingredient, one or more selected from the group consisting of a culture of the Phaeodactylum tricornutum TwOSC4 mutant of claim 1, a dried product thereof, and an extract thereof, or friedelin isolated from the mutant.

10. A cosmetic composition comprising, as an active ingredient, one or more selected from the group consisting of a culture of the Phaeodactylum tricornutum TwOSC4 mutant of claim 1, a dried product thereof, and an extract thereof, or friedelin isolated from the mutant.

11. A food composition comprising, as an active ingredient, one or more selected from the group consisting of a culture of the Phaeodactylum tricornutum TwOSC4 mutant of claim 1, a dried product thereof, and an extract thereof, or friedelin isolated from the mutant.

12. A health functional food comprising, as an active ingredient, one or more selected from the group consisting of a culture of the Phaeodactylum tricornutum TwOSC4 mutant of claim 1, a dried product thereof, and an extract thereof, or friedelin isolated from the mutant.

13. The composition of claim 9, wherein the composition is used for anti-inflammatory or antioxidant purposes.
